SEQUENCE LISTING (10 sequences)

SEQ ID NO: 1 - DNA, 585 bp, synthetic construct
  source 1..585: mol_type = other DNA
atggatttcatcctgaatatcagtatgaaaatggaagtgattttcaagaccgatctgcgcagtagtagccaggtggtttttcatgccggtagcctgtataattggtttagtgttgaaattatcaacagcggtcgcattgtgaccaccgcaattaaaaccctgctgagcaccgttaaatatgatattgtgaaaagcgcacgcatttatgccggtcagggctataccgaacatcaggcccaggaagaatggaatatgattctgcatgtgctgtttgaagaagaaaccgaaagtagcgcaagtagcgaaaatattcatgaaaaaaacgacaacgagaccaatgaatgtaccagcagctttgaaaccctgtttgaacaggaaccgagcagcgaagttccgaaagatagcaaactgtatatgctggcccagaaaaccgtgcagcatattgaacagtatggtaaagccccggattttaataaagttattcgcgcacataacttcattcagaccatttatggtaccccgctgaaagaagaagaaaaagaagtggttcgtctgatggtgattaaactgctgaaaaaaaaataa

SEQ ID NO: 2 - protein, 194 aa, synthetic construct
MDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKK

SEQ ID NO: 3 - DNA, 447 bp, synthetic construct
  source 1..447: mol_type = other DNA
aaaaaacagcagccgccgaaaaaagtctgcaaagtcgacaaagattgcggtagcggcgaacattgtgttcgcggtagttgtagcagtctgagctgtctggacgccgtcaaaatggacaaacgcaacatcaaaatcgacagcaaaatcagcagctgcgaatttaccccgaacttctaccgttttaccgataccgcagcggacgaacaacaagaattcggcaaaacccgccatccgatcaaaattaccccgagtccgtctgaaagccatagtccgcaggaagtctgcgaaaaatactgctcttggggtaccgacgattgtaccggttgggaatacgttggcgacgaaaaagaaggcacctgttacgtgtacaacaatccgcatcatccggtgctgaaatacggcaaagaccatatcatcgcgctgccgcgtaatcataaacacgcatga

SEQ ID NO: 4 - protein, 148 aa, synthetic construct
KKQQPPKKVCKVDKDCGSGEHCVRGSCSSLSCLDAVKMDKRNIKIDSKISSCEFTPNFYRFTDTAADEQQEFGKTRHPIKITPSPSESHSPQEVCEKYCSWGTDDCTGWEYVGDEKEGTCYVYNNPHHPVLKYGKDHIIALPRNHKHA

SEQ ID NO: 5 - DNA, 27 bp, synthetic construct
  source 1..27: mol_type = other DNA
ggatccatggatttcatcctgaatatc

SEQ ID NO: 6 - DNA, 26 bp, synthetic construct
  source 1..26: mol_type = other DNA
ctcgagtttttttttcagcagtttaa

SEQ ID NO: 7 - DNA, 25 bp, synthetic construct
  source 1..25: mol_type = other DNA
ggatccaaaaaacagcagccgccga

SEQ ID NO: 8 - DNA, 26 bp, synthetic construct
  source 1..26: mol_type = other DNA
ctcgagttatgcgtgtttatgattac

SEQ ID NO: 9 - DNA, 585 bp, synthetic construct
  source 1..585: mol_type = other DNA
atggattttattttaaatatatccatgaaaatggaggtcatcttcaaaacggatttaagatcatcttcacaagttgtgtttcatgcgggtagcctgtataattggttttctgttgagattatcaatagcggtagaattgttacgaccgctataaaaacattgcttagtactgttaagtatgatattgtgaaatctgctcgtatatatgcagggcaagggtatactgaacatcaggctcaagaagaatggaatatgattctgcatgtgctgtttgaagaggagacggaatcctcagcatcttcggagaacattcatgaaaaaaatgataatgaaaccaatgaatgcacatcctcctttgaaacgttgtttgagcaagagccctcatcggaggtacctaaagactccaagctgtatatgcttgcacaaaagactgtgcaacatattgaacaatatggaaaggcacctgattttaacaaggttattagagcacataattttattcaaaccatttatggaacccctctaaaggaagaagaaaaagaggtggtaagactcatggttattaaacttttaaaaaaaaaataa

SEQ ID NO: 10 - DNA, 534 bp, synthetic construct
  source 1..534: mol_type = other DNA
atgtttaatattaaaatgacaatttctacattgcttattgctcttattatactacttattattattttagtagtgtttttatactataagaaacaacaaccaccgaaaaaggtctgtaaagtagataaagattgtggtagtggagagcattgtgttcgtggatcatgtagctcattgagctgcttagatgccgtaaaaatggacaaacgaaatattaagatagattctaagatttcctcatgcgaattcactcccaatttttaccgttttacggatactgctgctgatgagcagcaagaatttggaaaaacacggcatcctataaaaataactccatctccaagtgaatcccatagcccccaagaggtgtgtgaaaaatattgttcatggggaaccgatgactgtacaggttgggaatatgttggtgatgaaaaggagggaacatgttatgtatataataatccacatcacccggttcttaaatatggtaaggatcacatcatagccttacctagaaatcataaacatgcataa

(19) United States
(12) Patent Application Publication
(10) Pub. No.: US 2023/0375548 A1
(43) Pub. Date: Nov. 23, 2023

(54) INDIRECT ENZYME-LINKED IMMUNOSORBENT ASSAY DETECTION KIT BASED ON P30 PROTEIN AND P22 PROTEIN OF AFRICAN SWINE FEVER VIRUS

(71) Applicant: Institute of Animal Science and Veterinary Medicine, Shandong Academy of Agricultural Sciences, Jinan (CN)

(72) Inventors: Jiang YU, Jinan (CN); Jianda LI, Jinan (CN); Jiaqiang WU, Jinan (CN); Yuyu ZHANG, Jinan (CN); Sufang REN, Jinan (CN); Fei LIU, Jinan (CN); Lin ZHANG, Jinan (CN); Wenbo SUN, Jinan (CN); Zhi CHEN, Jinan (CN); Luogang DING, Jinan (CN); Lihui GUO, Jinan (CN)

(21) Appl. No.: 18/318,996

(22) Filed: May 17, 2023

(30) Foreign Application Priority Data
May 23, 2022 (CN) 202210560778.2

(51) Int. Cl. G01N 33/569; G01N 33/68

(52) U.S. Cl. CPC G01N 33/56983; G01N 33/68; G01N 2469/00

(57) ABSTRACT

Disclosed is an indirect enzyme-linked immunosorbent assay (ELISA) detection kit based on p30 protein and p22 protein of African swine fever (ASF) p30 and p22, belonging to the technical field of animal biological products manufacturing. The indirect ELISA detection kit includes the p30 protein with an amino acid sequence as shown in SEQ ID NO: 2 and the p22 protein with an amino acid sequence as shown in SEQ ID NO: 4.

### CROSS-REFERENCE TO RELATED APPLICATIONS

This application claims priority to Chinese Patent Application No. 202210560778.2, filed on May 23, 2022, the contents of which are hereby incorporated by reference.

### INCORPORATION BY REFERENCE STATEMENT

This statement, made under Rules 77(b)(5)(ii) and any other applicable rule, incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: 20230512_sequence_347_005_2023_13228
Creation date: 12 May 2023
Byte size: 12,426

### TECHNICAL FIELD

The present application relates to the technical field of animal biological products manufacturing, and in particular to an indirect enzyme-linked immunosorbent assay detection kit based on p30 protein and p22 protein of African swine fever virus.

### BACKGROUND

African swine fever (ASF), as a virulent disease of swine caused by African swine fever virus (ASFV) infection, has brought a devastating shock to the farming industry. ASF is infectious to both domestic swine and wild boars of all ages with a short pathogenesis and a mortality rate of up to 100 percent (%), causing swine to die within 5-14 days after infection.

Currently, one of the effective strategies to control ASF is to isolate and eliminate infected animals, which requires highly sensitive and specific diagnostic analyses to rapidly detect ASFV-infected swine. In this regard, enzyme-linked immunosorbent assay (ELISA) is assigned under World Organization for Animal Health (OIE) regulations to detect the specific antibody of ASFV for international trade. The ELISA method is sensitive, simple, and economical diagnostic technique, which is expected to screen out ASFV antigen with good reactivity. Therefore, the development of an effective ELISA detection method is of paramount importance to prevent, control and eliminate ASF.

At present, there are several methods available for ASFV antigen detection, including fluorescence quantitative polymerase chain reaction (qPCR), recombinase polymerase amplification (RPA), microfluidic chip and others. But the methods for antibody detection are relatively limited with a predominant ELISA test kits and colloidal gold test strips. The commercialized ELISA test kits for ASF mainly encapsulate p72, p30, p54 and pp62. Of which p30 and p54 proteins are expressed and secreted in the early stage of ASFV infection, and p72 and pp62 proteins are expressed in the late stage of ASFV infection. Researches showed that among the three proteins of p30, p54 and p72, p30 induces the highest level of antibody expression. Although commercialized ELISA kits are mostly developed based on p30 protein, a kind of phosphorylated protein expressed early in infected cells, the accuracy of which needs to be further evaluated.

### SUMMARY

The present application provides an indirect enzyme-linked immunosorbent assay (ELISA) detection kit based on p30 protein and p22 protein of African swine fever virus (ASFV), to solve the problems existing in the prior art. The kit provided by the present application effectively detects the ASF in terms of antibody level.

To achieve the above objectives, the present application provides the following technical schemes:
an application of p30 protein and p22 protein in preparing an indirect ELISA detection kit for detecting ASF, where the p30 protein has an amino acid sequence as shown in SEQ ID NO: 2, and the p22 protein has an amino acid sequence as shown in SEQ ID NO: 4.

The present application also provides an indirect ELISA detection kit for detecting ASF, where the detection kit includes the p30 protein with the amino acid sequence as shown in SEQ ID NO: 2, and the p22 protein with the amino acid sequence as shown in SEQ ID NO: 4.

Optionally, the p30 protein has a coding gene as shown in SEQ ID NO: 1, and the p22 protein has a coding gene as shown in SEQ ID NO: 3.

Optionally, the p30 protein and the p22 protein are in a coating ratio of 1:3.

Optionally, the p30 protein is blocked using a sealing solution of 5 weight percentage (wt %) skimmed milk for 90 minutes (min); and the p22 protein is sealed with a sealing solution of 5 wt % skimmed milk for 60 min.

The present application discloses the following technical effects:

It is found that the p22 protein of ASFV, a 22 KiloDalton (kD) protein encoded by the KP177R gene, is a viral transmembrane structural protein that is transcribed early after ASFV infection and is located in the inner membrane of the viral particle and on the surface of infected cells. The p22 protein is highly conserved among the Eurasian strains of ASFV, and is relatively difficult to express because of the presence of the transmembrane structure. The structure and mechanism of the p22 protein remains largely unexplored, resulting in limited research on this protein. At present, the commercialized kits based on p30 protein are more developed for ASFV detection. However, achieving soluble expression of the p22 protein is relatively challenging due to its transmembrane region, hence there are no indirect ELISA kits based on p22 protein. Moreover, the compliance rate of the indirect ELISA kits for p22 protein alone with the existing kits is less than 90% after comparison.

The ASF antibody detection method for detecting two early secreted proteins of p30 and p22 at the same time improves the accuracy of the existing methods. According to the present application of indirect ELISA detection kit based on p30 protein and p22 protein, both recombinant proteins of p30 and p22 can react with ASF positive serum, and both show good reactivity. In addition, ASFV-positive serum remains positive after 12,800 dilutions and has a high sensitivity. The ELISA method of the present application is strongly specific to the ASF-positive serum and has no cross-reactivity with positive serum of porcine circovirus diseases (PCVD), pseudorabies, porcine reproductive and respiratory syndrome (PRRS), swine fever and Glasser's disease. The positive serum is selected for testing with inter-batch coefficients of variation (CV) ranging from 2.0% to 4.5% and intra-batch CV ranging from 3.0% to 6.0%, indicating that the kit of the present application has good repeatability.

### BRIEF DESCRIPTION OF THE DRAWINGS

In order to illustrate the technical solutions more clearly in the embodiments of the present application or in the prior art, the following is a brief description of the accompanying drawings to be used in the embodiments. It is obvious that the accompanying drawings in the following description are only some of the embodiments of the present application, and other drawings can be obtained according to them without any creative work for a person of ordinary skill in the art.

FIG. 1 shows exploration results of Isopropyl β-D-Thiogalactoside (IPTG) concentration as exploring p22 protein induction conditions. M represents protein molecular quality standard. 1 represents pET-32a empty vector. 2 represents pET-32a-p22 uninduced. 3-6 represent IPTG concentrations of 0.1 millimolar (mM), 0.4 mM, 0.7 mM, 1.0 mM, respectively.

FIG. 2 shows the exploration results of OD value. M represents protein molecular quality standard. 1 represents pET-32a empty vector. 2 represents pET-32a-p22 uninduced. 3-6 represent OD values of 0.4, 0.6, 0.8 and 1, respectively.

FIG. 3 illustrates exploration results of IPTG concentration as exploring p22 protein induction conditions. M represents protein molecular quality standard. 1 represents pET-32a empty vector. 2 represents pET-32a-p30 uninduced. 3-6 represent IPTG concentrations of 0.1 mM, 0.4 mM, 0.7 mM, 1.0 mM, respectively.

FIG. 4 shows the exploration results of OD value. M represents protein molecular quality standard. 1 represents pET-32a empty vector. 2 represents pET-32a-p30 uninduced. 3-6 represent OD values of 0.4, 0.6, 0.8 and 1, respectively.

FIG. 5 shows the reactivity of recombinant proteins p30. M represents protein molecular quality standard. 1 represents p30 protein.

FIG. 6 illustrate the reactivity of recombinant proteins p22. M represents protein molecular quality standard. 1 represents p22.

FIG. 7 shows the best protein coating concentrations and best serum dilutions of p30 protein.

FIG. 8 the best protein coating concentrations and best serum dilutions of p22 protein.

FIG. 9 shows the best blocking solutions and best blocking durations of p30 protein.

FIG. 10 shows the best blocking solutions and best blocking durations of p22 protein.

FIG. 11 is a schematic diagram illustrating a process of splicing and codon optimization of a gene sequence encoding p22, of which Majority is the gene sequence before modification as shown in SEQ ID NO: 10, and HLJ-2018-p22.seq P22-youhua.seq. is the modified gene sequence as shown in SEQ ID NO: 3.

FIG. 12 is a schematic diagram illustrating a process of codon optimization of a gene sequence encoding p30, of which Majority is the gene sequence before modification as shown in SEQ ID NO: 1, and HLJ-2018-p30.seq P30-youhua.seq. is the modified gene sequence as shown in SEQ ID NO: 9.

### DETAILED DESCRIPTION OF THE EMBODIMENTS

Various exemplary embodiments of the present application are now described in detail, and this detailed description should not be considered as a limitation of the present application, but should be understood as a more detailed description of certain aspects, features and embodiments of the present application.

It is to be understood that the terms described in the present application are intended to describe particular embodiments only and are not intended to limit the present application. Further, for the range of values in the present application, it is to be understood that each intermediate value between the upper and lower limits of the range is also specifically disclosed. Each smaller range between any stated value or intermediate value within a stated range, and any other stated value or intermediate value within a stated range is also included in the present application. The upper and lower limits of these smaller ranges may be independently included or excluded from the scope.

Unless otherwise stated, all technical and scientific terms used herein have the same meanings commonly understood by those of ordinary skill in the field to which this application relates. Although the present application only describes preferred methods and materials, any methods and materials similar or equivalent to those described herein can be used in the practice or testing of the present application. All documents mentioned in this specification are incorporated by reference to disclose and describe the methods and/or materials related to the documents. In case of conflict with any incorporated documents, the contents of this specification shall prevail.

Without departing from the scope or spirit of the present invention, it is obvious to those skilled in the art that many modifications and changes can be made to the specific embodiments of the present specification. Other embodiments obtained from the description of the present application will be obvious to the skilled person. The description and embodiment of the present application are only exemplary.

As used in this specification, the terms “comprising”, “including”, “having” and “containing” are all open terms, meaning including but not limited to.

The pET-32a vector used in the following embodiments is purchased from Wuhan Dai-An Biotechnology Co., Ltd.

### Embodiment 1

1. Construction and Synthesis of Recombinant Positive Plasmid

The gene sequences encoding p30 and p22 of African swine fever virus (ASFV) Pig/HLJ/2018, after splicing and codon optimization, is synthesized by Shanghai Generay Bioengineering Co., Ltd. and connected to the pEASY-Blunt vector, with those identified correctly by sequencing are the p30 and p22 gene positive cloning vectors.

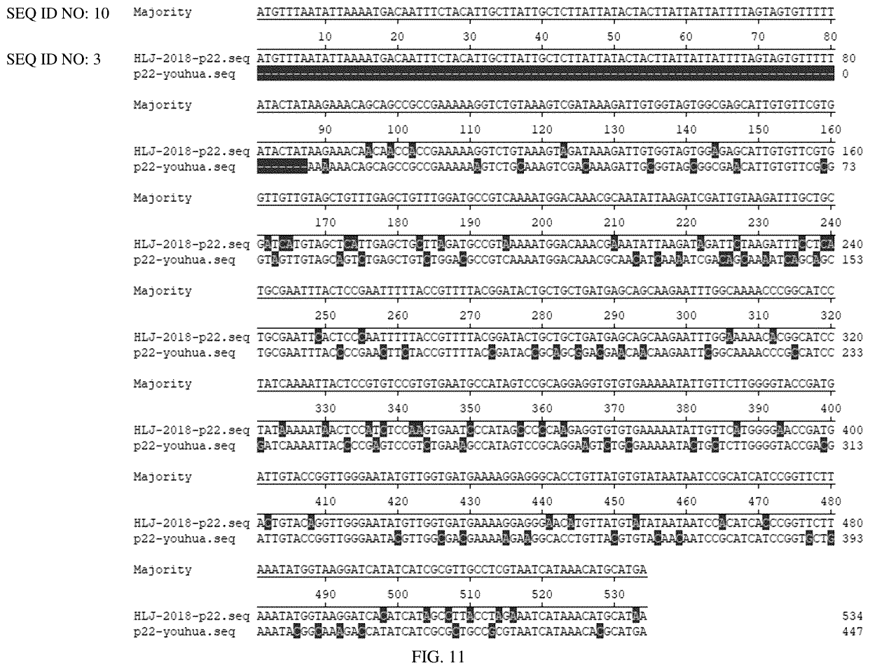

Process of splicing and codon optimization of the gene sequence encoding p22: the transmembrane region (the first 29 amino acids) is spliced out with reference to FIG. 11 for the specific nucleotide optimization.

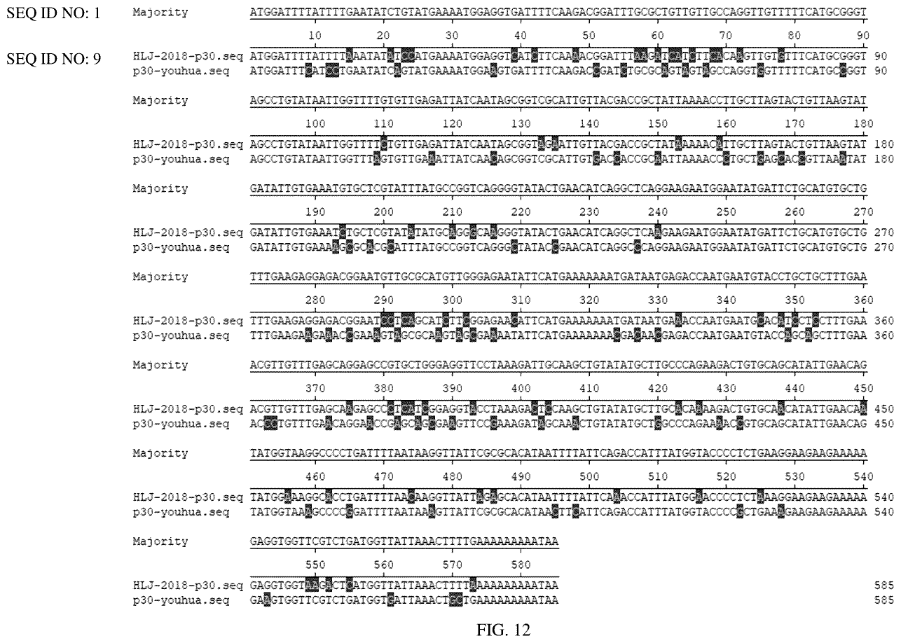

Process of codon optimization of the gene sequence encoding p30 with reference to FIG. 12 for the specific nucleotide optimization.

The positive cloned plasmids of p30 and p22 are double cleaved by restriction endonucleases BamHI and XhoI, respectively, and separately ligated with pET-32a vector that is also double cleaved, then the ligated products are transformed into the receptor cells DH5a, followed by sequencing by Sangon Biotech (Shanghai) Co., Ltd., where two recombinant positive plasmids with correct sequence identification are named as pET32a-p30 and pET32a-p22 respectively.

Specific primers of p30 and p22 genes are designed, with primer sequences as follows:

p30 upstream primer (SEQ ID NO: 5):
GGATCCATGGATTTCATCCTGAATATC (BamHI);
p30 downstream primer (SEQ ID NO: 6):
CTCGAGTTTTTTTTTCAGCAGTTTAA (XhoI);
p22 upstream primer (SEQ ID NO: 7):
GGATCCAAAAAACAGCAGCCGCCGA (BamHI);
and
p22 upstream primer (SEQ ID NO: 8):
CTCGAGTTATGCGTGTTTATGATTAC (XhoI).

The italicized underlined parts above indicate the enzyme cleavage sites.

The nucleotide sequence of p30 after codon optimization is as follows (SEQ ID NO: 1):

ATGGATTTCATCCTGAATATCAGTATGAAAATGGAAGTGATTTTCAAGAC
CGATCTGCGCAGTAGTAGCCAGGTGGTTTTTCATGCCGGTAGCCTGTATA
ATTGGTTTAGTGTTGAAATTATCAACAGCGGTCGCATTGTGACCACCGCA
ATTAAAACCCTGCTGAGCACCGTTAAATATGATATTGTGAAAAGCGCACG
CATTTATGCCGGTCAGGGCTATACCGAACATCAGGCCCAGGAAGAATGGA
ATATGATTCTGCATGTGCTGTTTGAAGAAGAAACCGAAAGTAGCGCAAGT
AGCGAAAATATTCATGAAAAAAACGACAACGAGACCAATGAATGTACCAG
CAGCTTTGAAACCCTGTTTGAACAGGAACCGAGCAGCGAAGTTCCGAAAG
ATAGCAAACTGTATATGCTGGCCCAGAAAACCGTGCAGCATATTGAACAG
TATGGTAAAGCCCCGGATTTTAATAAAGTTATTCGCGCACATAACTTCAT
TCAGACCATTTATGGTACCCCGCTGAAAGAAGAAGAAAAAGAAGTGGTTC
GTCTGATGGTGATTAAACTGCTGAAAAAAAAATAA.

The amino acid sequence of p30 is as follows (SEQ ID NO: 2):

MDFILNISMKMEVIFKTDLRSSSQVVFHAGSLYNWFSVEIINSGRIVTTA
IKTLLSTVKYDIVKSARIYAGQGYTEHQAQEEWNMILHVLFEEETESSAS
SENIHEKNDNETNECTSSFETLFEQEPSSEVPKDSKLYMLAQKTVQHIEQ
YGKAPDFNKVIRAHNFIQTIYGTPLKEEEKEVVRLMVIKLLKKK.

The nucleotide sequence of p22 after codon optimization is as follows (SEQ ID NO: 3):

AAAAAACAGCAGCCGCCGAAAAAAGTCTGCAAAGTCGACAAAGATTGCGG
TAGCGGCGAACATTGTGTTCGCGGTAGTTGTAGCAGTCTGAGCTGTCTGG
ACGCCGTCAAAATGGACAAACGCAACATCAAAATCGACAGCAAAATCAGC
AGCTGCGAATTTACCCCGAACTTCTACCGTTTTACCGATACCGCAGCGGA
CGAACAACAAGAATTCGGCAAAACCCGCCATCCGATCAAAATTACCCCGA
GTCCGTCTGAAAGCCATAGTCCGCAGGAAGTCTGCGAAAAATACTGCTCT
TGGGGTACCGACGATTGTACCGGTTGGGAATACGTTGGCGACGAAAAAGA
AGGCACCTGTTACGTGTACAACAATCCGCATCATCCGGTGCTGAAATACG
GCAAAGACCATATCATCGCGCTGCCGCGTAATCATAAACACGCATGA.

The amino acid sequence of p22 is as follows (SEQ ID NO: 4):

KKQQPPKKVCKVDKDCGSGEHCVRGSCSSLSCLDAVKMDKRNIKIDSKIS
SCEFTPNFYRFTDTAADEQQEFGKTRHPIKITPSPSESHSPQEVCEKYCS
WGTDDCTGWEYVGDEKEGTCYVYNNPHHPVLKYGKDHIIALPRNHKHA.

p30 original sequence (SEQ ID NO: 9):

ATGGATTTTATTTTAAATATATCCATGAAAATGGAGGTCATCTTCAAAAC
GGATTTAAGATCATCTTCACAAGTTGTGTTTCATGCGGGTAGCCTGTATA
ATTGGTTTTCTGTTGAGATTATCAATAGCGGTAGAATTGTTACGACCGCT
ATAAAAACATTGCTTAGTACTGTTAAGTATGATATTGTGAAATCTGCTCG
TATATATGCAGGGCAAGGGTATACTGAACATCAGGCTCAAGAAGAATGGA
ATATGATTCTGCATGTGCTGTTTGAAGAGGAGACGGAATCCTCAGCATCT
TCGGAGAACATTCATGAAAAAAATGATAATGAAACCAATGAATGCACATC
CTCCTTTGAAACGTTGTTTGAGCAAGAGCCCTCATCGGAGGTACCTAAAG
ACTCCAAGCTGTATATGCTTGCACAAAAGACTGTGCAACATATTGAACAA
TATGGAAAGGCACCTGATTTTAACAAGGTTATTAGAGCACATAATTTTAT
TCAAACCATTTATGGAACCCCTCTAAAGGAAGAAGAAAAAGAGGTGGTAA
GACTCATGGTTATTAAACTTTTAAAAAAAAAATAA.

p22 original sequence (SEQ ID NO: 10):

ATGTTTAATATTAAAATGACAATTTCTACATTGCTTATTGCTCTTATTAT
ACTACTTATTATTATTTTAGTAGTGTTTTTATACTATAAGAAACAACAAC
CACCGAAAAAGGTCTGTAAAGTAGATAAAGATTGTGGTAGTGGAGAGCAT
TGTGTTCGTGGATCATGTAGCTCATTGAGCTGCTTAGATGCCGTAAAAAT
GGACAAACGAAATATTAAGATAGATTCTAAGATTTCCTCATGCGAATTCA
CTCCCAATTTTTACCGTTTTACGGATACTGCTGCTGATGAGCAGCAAGAA
TTTGGAAAAACACGGCATCCTATAAAAATAACTCCATCTCCAAGTGAATC
CCATAGCCCCCAAGAGGTGTGTGAAAAATATTGTTCATGGGGAACCGATG
ACTGTACAGGTTGGGAATATGTTGGTGATGAAAAGGAGGGAACATGTTAT
GTATATAATAATCCACATCACCCGGTTCTTAAATATGGTAAGGATCACAT
CATAGCCTTACCTAGAAATCATAAACATGCATAA.

2. Induced Expression and Protein Purification of Recombinant Proteins p30 and p22

The specific operation of induction includes:

The two recombinant plasmids, pET32a-p30 and pET32a-p22, are transformed into competent cells BL21 respectively, then a single colony is picked out from a solid medium cultured overnight in a 37 degree Celsius (° C.) incubator and put into a Luria-Bertani (LB) liquid medium with ampicillin (Amp) resistance, followed by culturing overnight at 37° C. and 200 revolutions per minute (rpm). 50 microliters (μL) of the bacterial solutions of pET 32a-P30, pET32a-p22 and pET-32a are taken respectively and placed in 3 test tubes containing 5 milliliters (mL) of LB liquid medium (Amp+). Then a tube of 5 mL LB liquid medium (Amp+) without bacterial solution is set as a negative control, followed by culturing on a constant temperature shaking table at 37° C. and 200 rpm for 3-4 hours (h) so that the two bacterial solutions can reach OD₆₀₀ of between 0.6-0.8. 500 μL of bacterial solution is taken form the three tubes with bacterial solution as the control before induction and stored at 4° C. Then the three tubes with remained bacterial solution are each added with 7.5 μL of 100 mM Isopropyl β-D-Thiogalactoside (IPTG) solution, followed by culture at 37° C. and 200 rpm for 4 h, and 500 μL of bacterial solution is taken from each tube as the bacteria solutions after induction. The three bacteria solutions before induction and three bacteria solutions after induction are centrifuged (9,000 rpm, 4° C.) for 10 min, then the supernatant discarded and the sludge is left. All bacteria solutions before induction and after induction are resuspended with 100 μL phosphate buffer saline (PBS), and then centrifuged at 9,000 rpm at 4° C. for 10 min, the supernatants are discarded with bacteria sludge being left; the above steps are repeated twice; and 25 μL of 5×SDS-PAGE sample buffer is added into each final suspended bacterial solution. All bacteria samples are boiled in boiling water for 10 min and stored at −20° C., and subjected to SDS-PAGE gel electrophoresis detection.

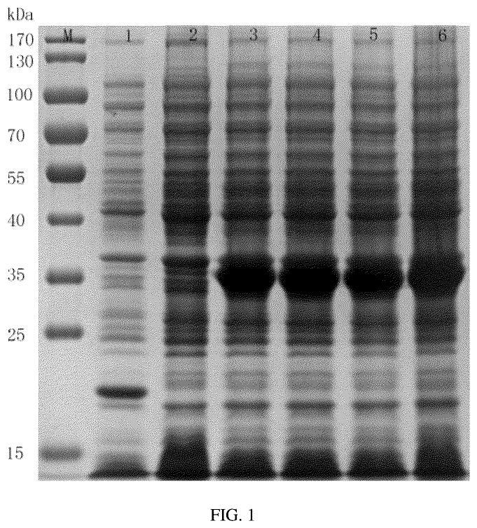

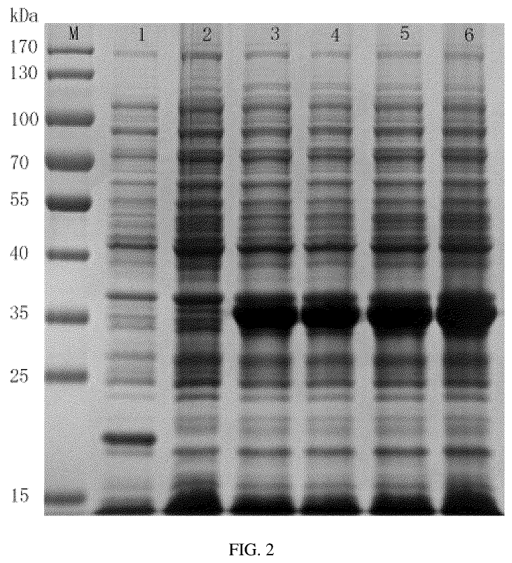

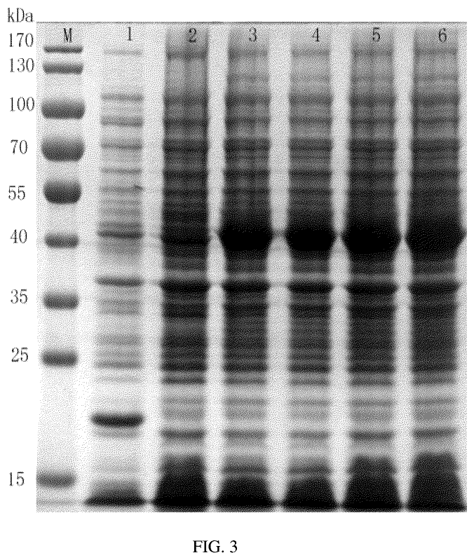

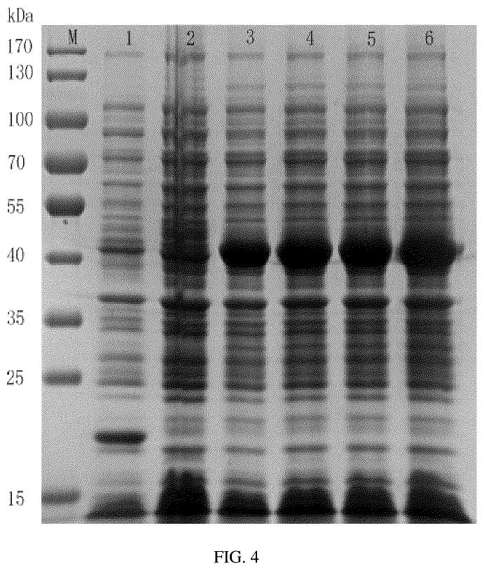

By exploring the induction expression conditions (IPTG concentration, OD value, induction temperature, induction time), the best expression conditions are determined (as shown in FIG. 1-FIG. 4). After the target proteins p30 and p22 are induced by IPTG, target proteins of single band are obtained by purification with nickel column, and the concentrations of the purified two recombinant proteins are determined by bicinchoninic acid (BCA) protein quantitative kit, with result of recombinant p30 as 0.04 gram per liter (g/L) and recombinant p22 as 0.334 g/L.

3. Identification of Reactivity of Recombinant Proteins p30 and p22

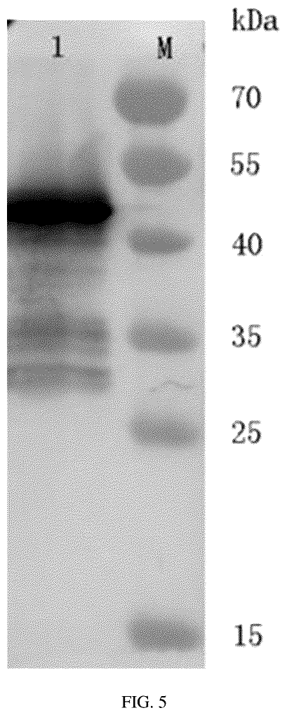

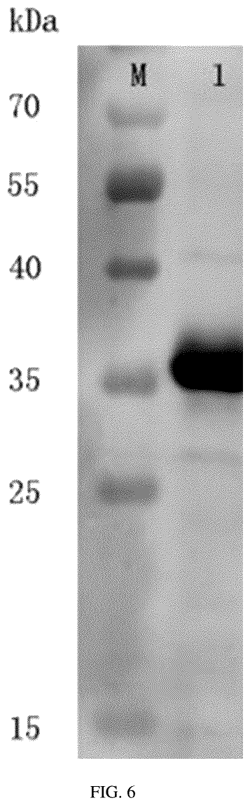

The purified recombinant proteins p30 and p22 are used as antigens, ASF positive serum is used as primary antibody, goat anti-pig (goat pAb to pig IgG) antibody conjunct-HRP is used as secondary antibody, which are subjected to western blot analysis. The results show that the bands of p30 and p22 are clear and single, and are consistent with the expected size, indicating that both recombinant proteins p30 and p22 can react with ASF positive serum, and both of them have good reactivity (FIG. 5 and FIG. 6).

4. Optimization of Reaction Conditions of Enzyme-Linked Immunosorbent Assay (ELISA) Method

The kit includes: protein coated buffer, phosphate buffered saline with tween (PBST) solution, blocking solution, sample diluent, enzyme-labeled secondary antibody, tetramethyl benzidine (TMB) single-component chromogenic solution and stop buffer.

4.1 Optimum Protein Coating Concentration and Serum Dilution

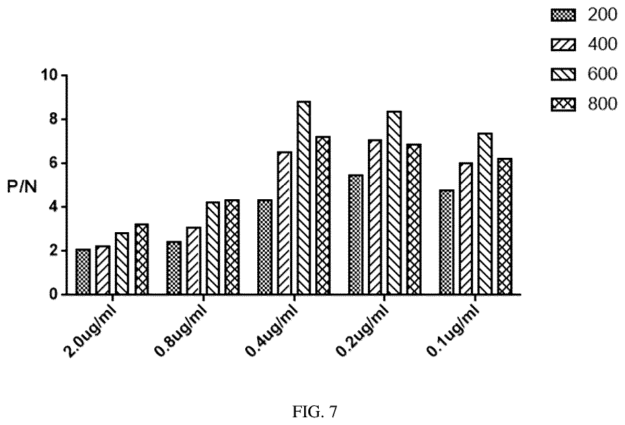

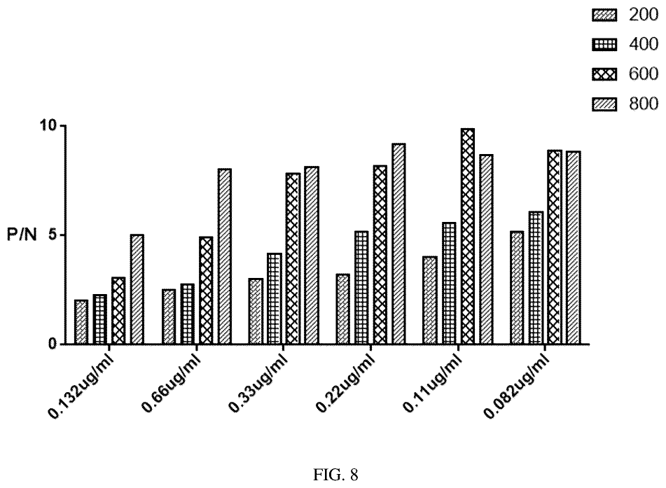

By the method of checkerboard titration, the optimal coating concentration of p30 protein is determined to be 0.4 milligram per liter (mg/L), the optimal dilution of sample serum is 600 times, the optimal coating concentration of p22 protein is 0.11 mg/L, and the optimal dilution of serum sample is 600 times (FIG. 7 and FIG. 8).

4.2 Determination of the Optimal Blocking Liquid and Blocking Duration

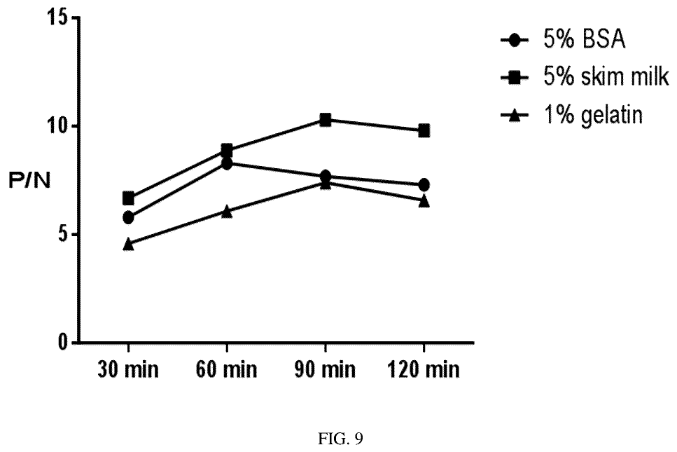

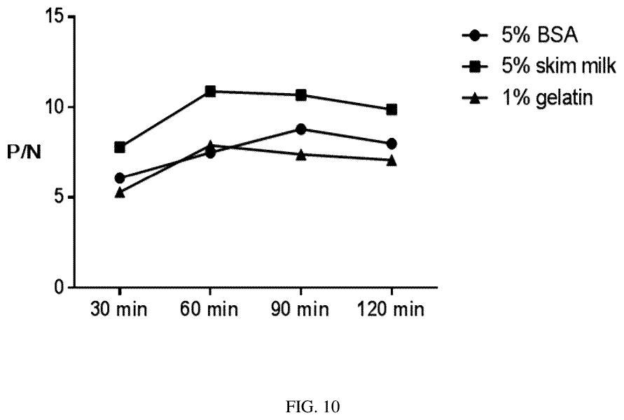

After the optimal protein coating concentration and serum dilutions are determined, the above steps are repeated, with test results showing that the best blocking solution for p30 protein is 5 percent (%) skimmed milk, and the best blocking duration is 90 min, while the best blocking solution for p22 protein coated plate is 5% skimmed milk, and the best blocking duration is 60 min (FIG. 9 and FIG. 10).

4.3 Determination of the Optimal Coating Ratio of p30 and p22

p30 and p22 proteins (according to the optimal coating concentration) are coated with enzyme-labeled plates at the volume ratio of 1:1, 1:2, 2:1, 1:3 and 3:1 (total volume of 100 mL), respectively; the optimal coating ratio of two protein is the maximum P/N value; and the experimental results show that when the ratio of p30 to p22 is 1:3, P/N is the highest (Table 1).

TABLE 1
Coating volume ratio of p30 and p22

| Volume ratio of p30 to p22 | p30 | p22 | 1:1 | 2:1 | 1:2 | 3:1 | 1:3 |
|---|---|---|---|---|---|---|---|
| ASFV-positive sera | 2.920 | 1.394 | 1.537 | 1.672 | 1.261 | 1.502 | 0.835 |
| ASFV-negative sera | 0.474 | 0.195 | 0.244 | 0.219 | 0.164 | 0.186 | 0.101 |
| P/N | 6.2 | 7.2 | 6.3 | 7.6 | 7.7 | 8.1 | 8.3 |

4.4 Determination of Critical Value

According to the above optimal conditions, 50 negative serum (finished product kit test) are selected for ELISA test and the values of OD₄₅₀ are read to calculate the average value x̅ (0.174) and standard deviation s (0.083), where x̅+2S=0.34, and negative ≤0.34; x̅+3S=0.423, and positive ≥0.423; the middle range is suspicious.

4.5 Sensitivity Test

ASF positive serum of different dilutions are detected by p30 protein indirect ELISA kit, p22 protein indirect ELISA kit, p30 and p22 dual protein indirect ELISA kit and two commercialized kits, respectively, with OD₄₅₀ being determined as well; the results as illustrated in Tables 2-5 show that the detection results of p30 and p22 coated plates are still positive (Table 3) after 12,800 times of dilutions of serum according to the critical value.

TABLE 2
Sensitivity test results of P30 protein indirect ELISA kit

| Dilutions | 200 | 400 | 800 | 1,600 | 3,200 | 6,400 | 12,800 | 25,600 |
|---|---|---|---|---|---|---|---|---|
| OD₄₅₀ value | 2.516 | 2.237 | 1.843 | 1.576 | 1.288 | 0.839 | 0.456 | 0.225 |
| P or N | + | + | + | + | + | + | + | − |

TABLE 3
Sensitivity test results of p22 protein indirect ELISA kit

| Dilutions | 200 | 400 | 800 | 1,600 | 3,200 | 6,400 | 12,800 | 25,600 |
|---|---|---|---|---|---|---|---|---|
| OD₄₅₀ value | 2.000 | 1.859 | 1.614 | 1.177 | 0.854 | 0.584 | 0.35 | 0.196 |
| P or N | + | + | + | + | + | + | + | − |

TABLE 4
Sensitivity test results of p30 and p22 dual protein indirect ELISA kit

| Dilutions | 200 | 400 | 800 | 1,600 | 3,200 | 6,400 | 12,800 | 25,600 |
|---|---|---|---|---|---|---|---|---|
| OD₄₅₀ value | 2.387 | 2.105 | 1.791 | 1.379 | 1.086 | 0.732 | 0.435 | 0.216 |
| P or N | + | + | + | + | + | + | + | − |

TABLE 5
Sensitivity test results of commercialized kits

| Dilutions | 200 | 400 | 800 | 1,600 | 3,200 | 6,400 | 12,800 | 25,600 |
|---|---|---|---|---|---|---|---|---|
| OD₄₅₀ value | 0.119 | 0.171 | 0.295 | 0.410 | 0.544 | 0.782 | 1.123 | / |
| P or N | + | + | + | + | − | − | − | − |

4.6 Specificity Test

Specificity assessment of ELISA cross-reactivity tests for porcine circovirus diseases (PCVD), pseudorabies, porcine reproductive and respiratory syndrome (PRRS), swine fever and porcine Glasser's disease is performed. The results show that OD₄₅₀ of positive serum of the five swine diseases are all less than 0.34, indicating that the developed ELISA kit and proving that the method has good specificity (Table 6).

TABLE 6
Specificity test

| Indicators | | | | | | | Porcine |
|---|---|---|---|---|---|---|---|
| and | Negative | Positive | | | | Swine | Glasser's |
| results | sample | sample | PCVD | Pseudorabies | PRRS | fever | disease |
| OD₄₅₀ | 0.110 | 1.521 | 0.134 | 0.223 | 0.204 | 0.153 | 0.209 |
| Results | − | + | − | − | − | − | − |

4.7 Repeatability Test

Four kinds of positive serum selected, with intra-batch coefficients of variation (CV) ranging from 2.0% to 4.5%, and inter-batch coefficients of variation ranging from 3.0% to 6.0%, which prove that the established indirect ELISA kit has good repeatability (Table 7).

TABLE 7
Repeatability test

| | Intra-batch | | Inter-batch | |
|---|---|---|---|---|
| Sample | Result | CV % | Result | CV % |
| 1 | 2.513 ± 0.072 | 2.86 | 2.525 ± 0.135 | 5.34 |
| 2 | 1.404 ± 0.045 | 3.21 | 1.372 ± 0.025 | 2.55 |
| 3 | 1.64 ± 0.033 | 2.01 | 1.684 ± 0.054 | 3.21 |
| 4 | 0.835 ± 0.034 | 4.07 | 0.821 ± 0.39 | 4.75 |

4.8 Sample Detection

Three established indirect ELISA kits above are used to detect 184 swine blood samples. The results show 105 positive and 79 negative samples are detected by the established indirect ELISA kits based on p30 and p22 dual proteins. While the compliance rate of the established indirect ELISA kit based on p30 and p22 proteins with the commercial ELISA kit ID.vet is slightly lower at 94.6%, the established ELISA detects 2 more positive samples and 2 more suspicious samples, compared to the commercial ELISA kit 1 (ID.vet ELISA kit) The compliance rate of commercialized kit 2 with commercial kit 1 is 96.2%, and the results indicate that the detection rate of positive and suspicious samples of the established indirect ELISA kit based on p30 and p22 dual protein is higher than that of the two commercial kits (Table 8 and Table 9).

TABLE 8
Test results of different kit samples

| | | | Number of | | | Compliance |
|---|---|---|---|---|---|---|
| | Number | Number of | negative | Number of | Samples of | rate |
| | of | positive | samples | suspicious | different | (compared |
| Kit | samples | samples | detected | samples | results | with kit 1) |
| p30 and p22 | 184 | 101 | 79 | 4 | 10 | 94.6% |
| p30 | 184 | 96 | 82 | 6 | 9 | 95.1% |
| p22 | 184 | 86 | 95 | 3 | 23 | 87.5% |
| Commercial | 184 | 99 | 83 | 2 | / | / |
| kit 1 | | | | | | |
| Commercial | 184 | 96 | 87 | 1 | 7 | 96.2% |
| kit 2 | | | | | | |

TABLE 9
Statistical results of detection rate of different kit samples

| | | Positive samples | | Negative samples | |
|---|---|---|---|---|---|
| | | Number | Correct | Number | Correct |
| | | of | detection | of | detection |
| | Number | positive | rate of | negative | rate of |
| | of | samples | positive | samples | negative |
| Kit | samples | detected | samples | detected | samples |
| p30 and p22 | 184 | 101 | 54.9% | 79 | 42.9% |
| p30 | 184 | 96 | 52.2% | 82 | 44.6% |
| p22 | 184 | 86 | 46.7% | 95 | 51.6% |
| Commercialized kit 1 | 184 | 99 | 53.8% | 83 | 45.1% |
| Commercialized kit 2 | 184 | 96 | 52.2% | 87 | 47.3% |

The above-mentioned embodiments only describe the preferred mode of the present application, but do not limit the scope of the present application. On the premise of not departing from the design spirit of the present application, all kinds of modifications and improvements made by ordinary technicians in the field to the technical scheme of the present application shall fall within the scope of protection determined by the claims of the present application.

What is claimed is:

1. An application of p30 protein and p22 protein in preparing an indirect enzyme-linked immunosorbent assay (ELISA) detection kit for detecting African swine fever, comprising the p30 protein with an amino acid sequence as shown in SEQ ID NO: 2, wherein the p22 protein with an amino acid sequence is shown in SEQ ID NO: 4.

2. An ELISA detection kit for detecting African swine fever, comprising a p30 protein with an amino acid sequence as shown in SEQ ID NO: 2, and a p22 protein with an amino acid sequence as shown in SEQ ID NO: 4.

3. The detection kit according to claim 2, wherein the p30 protein has a coding gene as shown in SEQ ID NO: 1, and the p22 protein has a coding gene as shown in SEQ ID NO: 3.

4. The detection kit according to claim 2, wherein the p30 protein and the p22 protein are in a coating ratio of 1:3.

5. The detection kit according to claim 2, wherein a sealing solution for the p30 protein is 5 wt % skimmed milk with a sealing duration of 90 minutes, and a sealing solution for the p22 protein is 5 wt % skimmed milk with a sealing duration of 60 minutes.
